(19) United States
(12) Patent Application Publication
(10) Pub. No.: US 2021/0322988 A1
(43) Pub. Date: Oct. 21, 2021

(54) MICROFLUIDIC DROPLET-BASED ASSAY PROCESS AND APPARATUS

(71) Applicants: BIOMÉRIEUX, Marcy L'Etoile (FR); BIOASTER, Lyon (FR)

(72) Inventors: Christophe VEDRINE, Carrières-sur-Seine (FR); Yacine BOUNAB, Asnières-sur-Seine (FR)

(73) Assignees: BIOMÉRIEUX, Marcy L'Etoile (FR); BIOASTER, Lyon (FR)

(21) Appl. No.: 17/271,010

(22) PCT Filed: Sep. 23, 2019
(86) PCT No.: PCT/EP2019/075515

§ 371 Date: Feb. 24, 2021

(30) Foreign Application Priority Data
Sep. 25, 2018 (EP) 18306247.0

(51) Int. Cl. B01L 3/00; G01N 33/58; G01N 33/543; G01N 33/50; G01N 21/64

(52) U.S. Cl. CPC B01L 3/502792; G01N 33/582; G01N 33/54333; G01N 2021/6439; G01N 21/6428; B01L 2400/043; G01N 33/5044

(57) ABSTRACT

The invention provides for a microfluidic droplet-based assay process and apparatus, wherein the process comprises the step of creating several assay droplets (18) containing paramagnetic particles (22, 22′), detectable markers (24), and at least one biological cell (10), wherein the paramagnetic particles (22, 22′) and the detectable markers (24) are able to bind with a target analyte (16) released by the cell, wherein the process includes storing several assay droplets along a horizontal storage plane, includes the generation of a magnetic field through the stored assay droplets, and includes detecting a physical characteristic of the detectable markers contained in a given assay droplet by optical imaging of the detectable markers, characterized in that the generation of a magnetic field generates a magnetic field such that the paramagnetic particles are attracted towards a lateral and inferior portion of the given stored assay droplet in which they are contained.

The invention lies within the field of microfluidic droplet-based assay processes and of apparatus for analyzing biological cell activity by detection of at least one target analyte contained in one or several droplets.

One application of the invention relates to the characterization of cell secretion dynamics, i.e. the evolution of such secretion over time.

Studies have shown that cell secretion dynamics and their heterogeneity play a fundamental role in physiological and pathological processes. The characterization of heterogeneous dynamic responses at the single cell level is crucial for understanding biological systems and to design effective therapeutic interventions. For instance, dynamic profiling of cancer cell secretome has permitted the discovery of novel biomarkers for cancer diagnostics and to evaluate therapies. To protect the organism against invaders, the immune system mobilizes a set of dynamic processes that includes signaling molecule secretions, cell differentiation, cell migration as well as phagocytosis. Immune responses of individual cells to external cues such as, for instance, simple exposure to a homogeneous ligand, are highly dynamic and display dramatic cell-to-cell variations. One key feature in immune reaction is the production and secretion of a large panel of signaling cytokines that coordinate the immune functions. Deciphering cytokine dynamic processes is necessary to understand mechanisms underlying immunity and to help building predictive models for the immune system and future therapies. However, owing to limitations of current methodologies, little is known about cytokine dynamic profiles in the context of individual cells. The most commonly used technologies to study production and secretion of cytokines at single cell resolution are flow cytometry and Enzyme-linked immunospot (ELISPOT) assay, respectively. However, these single-time-point-based technologies do not enable full understanding of cell functions dynamics. In addition, these approaches use large volumes of both samples and assay reagents. The introduction of miniaturization of laboratory procedures has held great potential for single cell-based assays (see Hummer, D., Kurth, F., Naredi-Rainer, N. & Dittrich, P. S. “Single cells in confined volumes: microchambers and microdroplets”—Lab chip, 2016, 447-458, referred to hereinafter as “Hummer et al., 2016”). In particular, novel technologies, based on microfabrication and microfluidics, have been developed to assess cytokine secretion from single cells. Micro-engraving technology uses a dense array of polydimethylsiloxane (PDMS)-based nanowells for simultaneous dynamic measurements of multiple cytokines that are secreted by individual cells. It can be noted that this technology suffers from complicated device fabrication processes and, in some cases, complex handling of cells and assay reagents. In addition, non-specific absorption of secreted proteins by PDMS-based nanowells might affect the sensitivity and reproducibility of the assay. Also, single-cell barcode chip (SCBC) technology for multiplexed analysis and droplet microfluidics assays for high-throughput screening have already been developed for cytokine secretion. However, as previously mentioned, these endpoint approaches do not assess the dynamic nature of cytokine-secreting cells.

Thus, there remains the need for a simple and robust droplet-based microfluidic assay which, when combined with optical imaging, including for example, time-lapse fluorescence microscopy, enables assessing the functional dynamics of individual cells, for example of immune cells.

Single-cell biology assays can benefit considerably from the unique capabilities offered by droplet microfluidics systems (see Linas Mazutis, John Gilbert, W Lloyd Ung, David A Weitz, Andrew D Griffiths, John A Heyman—«Single-cell analysis and sorting using droplet-based microfluidics»—Nature Protocols, 8(5), 870-891 (2013), hereinafter referred to as “Mazutis et al, 2013”). The cell and bioassay reagents are confined into highly monodisperse droplets with volumes ranging from pico- to nanoliters. Each droplet serves as an independent bioreactor to assess biochemical cellular reactions, thus enabling ultrahigh-throughput screening and analysis of large numbers of single cells. Droplet microfluidics combined with time-lapse microscopy is suitable for fast and quantitative real-time detection of the molecule of interest secreted by the single cell (Mazutis et al., 2013; and Klaus Eyer, Raphaël C L Doineau, Carlos E Castrillon, Luis Briseño-Roa, Vera Menrath, Guillaume Mottet, Patrick Eng/and, Alexei Godina, Elodie Brient-Litz/er, Clément Nizak, Allan Jensen, Andrew D Griffiths, Jérôme Bibette, Pierre Bruhns & Jean Baudry: “Single-cell deep phenotyping of IgG-secreting cells for high-resolution immune monitoring”; published in NATURE BIOTECHNOLOGY, volume 35, number 10, October 2017, hereafter referred to as “Eyer et al, 2017”). The compartmentalization of single cells together with ultrasensitive bioassays into picoliter volumes accelerates detection of biological responses. Indeed, upon activation, the molecules secreted by individual cells rapidly accumulate within droplets and rapidly reach detectable concentrations; thus only a few molecules are needed to reach the limit of detection. For example, 3000 molecules are sufficient to reach a 0.1 nM limit of detection (LOD) in 50-pL droplet, which would be equivalent to 6×10⁹ molecules in the 100 μL of a microtiter-plate-based assay.

Another advantage of droplet-based assays is the possibility to fine-control the environment of the confined single cell and thus to assess the functional dynamics and diversity of cells in well-defined conditions. Moreover, microfluidic-based cell assays are an attractive analytical tool for working with low volume specimens obtained from biohazardous origins or with precious clinical samples (Mazutis et al., 2013).

Examples of microfluidic droplet-based assay processes and apparatus are described in a document by:
Jöensson, H., & Svahn, H. A. (2012)—“Droplet Microfluidics—A Tool for SingleCell Analysis”; published in Angewandte Chemie International Edition, 51(49), 12176-12192; DOI: 10.1002/anie.201200460; hereafter referred to as “Jöensson et al., 2012”
   and in a document by
Yu Fen Samantha Seah, Hongxing Hu, Christoph A. Merten, “Microfluidic single-cell technology in immunology and antibody screening”; published in Molecular Aspects of Medicine 59 (2018), 47-61; available at https://doi.org/10.1016/j.mam.2017.09.004; hereafter referred to as “Seah et al., 2019”

As an example of analyzing biological cell activity, here in relation to immune cell secretion dynamic, the inventors have devised a process and an apparatus to assess functional dynamic of primary human monocyte at single cell resolution. Monocytes are key cells of the innate immune system for inflammatory responses. Human monocytes represent 10% of circulating leukocytes and are well known to exhibit heterogeneity in term of morphology, surface markers, endocytosis activity as well as signaling molecules secretome.

The exemplary bioassay which will be detailed hereinafter is based on single monocyte compartmentalization in 50-pL droplets together with nanobeads binding assay to reveal cytokine secretion and with fluorescent probes to assess cell viability. Lipopolysaccharide (LPS) is also confined within the droplet to activate the monocyte and trigger the secretion of cytokines. Following the joint encapsulation of a cell and bioassay reagents, the resulting emulsion is transferred into a glass storage chamber. The chamber content is imaged with an automated fluorescence microscope at regular time interval, with an image resolution enabling analysis of single droplet content.

The inventors have successfully activated and monitored the time course behavior of a large number of single CD14 human monocytes. Cells remained alive within the droplets for over the course of the experiment and the ultrasensitive homogenous fluorescent-based binding assay enabled fast and rapid detection of TNF-alpha secretion dynamics at the single cell level. A 30 min stimulation at 37° C. was sufficient to detect secreted TNF-alpha from a single monocyte. Thus, the sensitivity of the process and apparatus according to the invention exceeds that of conventional single cell secretion assays such as ELISPOT. Unlike endpoint measurement assays, the inventor's approach provided quantitative measurement of TNF-alpha secretion rate, allowing a deep characterization of monocyte functional dynamic profiles. This multidimensional analysis revealed a complex picture of TNF-alpha secretion dynamics of single CD14 monocyte.

The process and apparatus could be used to assess other types of biological activity, including by analyzing secretion dynamics of signaling molecules and cell heterogeneity of a diverse range of both immune and non-immune cells in biological samples. This may have clinical diagnostic relevance for diseases associated with cell dynamic response and their heterogeneity.

In essence, the invention deals with a microfluidic droplet-based assay process for analyzing biological cell activity by detection of at least one target analyte contained in one or several droplets, and deals with an apparatus for implementing such a process. The process comprises the step of creating one or several assay droplets of a fluid, typically an aqueous liquid, more typically a liquid culture medium where an assay droplet contains:
paramagnetic particles;
detectable markers;
and, at least one biological cell potentially capable of releasing at least one target analyte.

The process relies on the paramagnetic particles and the detectable markers being able to bind with a target analyte to form a marked and magnetized combination with a target analyte. Then, several assay droplets are stored along a single layer, along a horizontal storage plane.

Such a process and apparatus are described in Eyer et al., 2017. This document describes such a process and apparatus which include the generation of a magnetic field through assay droplets stored in storage chamber for spatially arranging the paramagnetic particles inside the stored assay droplet. The process and apparatus use a detection method based on optical imaging. Optical imaging is performed along an imaging axis A1 (see FIG. 3), which is here substantially perpendicular to the horizontal storage plan, i.e. vertically. Spatially arranging the paramagnetic particles inside the droplets may include grouping the marked and magnetized combinations together geographically in the droplets, which may make the detectable markers more visible, due to a local higher concentration at the location where the marked and magnetized combinations are grouped together in the droplets.

Document WO 2016/059182 discloses a similar process and apparatus. With such a process and apparatus, wherein the magnetic field is generated by an assembly of two magnets which are located on two opposites sides of the storage chamber, the secretion of the target analyte is highlighted by the observation of a relocation and concentration of the fluorescence to form an elongated object, more or less fluorescent, extending in the droplet from one side to the other.

Although being very promising, the process and apparatus described in these two documents allow only a detection and/or a measurement of a florescence that is supposed to reflect the possible release of a target analyte. These described process and apparatus provide no information about the cells encapsulated in the monitored droplets, neither their number (possibly their absence) nor their physiological state, thenceforth the generated results are binary and very basic, which must necessarily come from the observation of a large number of droplets. A positive signal (i.e. a positive detection of a fluorescence a relocation and concentration of the fluorescence inside the droplet) recorded for a significant number of droplets is supposed to be associated with a positive detection of the targeted activity. A negative signal recorded with almost all the droplets is conversely associated with a non-response of the cell for the targeted activity.

Additionally, in the event that an exploitation of these processes and apparatus is wished in an optical imaging field, more particularly in cell imaging field, the magnetic field which is generated, here by an assembly of two magnets which are located on two opposite sides of the storage chamber, leads to some difficulties in obtaining proper and usable images. Indeed, in the process and the method, it has been found that it was difficult to obtain images in which both the cell and the detectable markers could be pictured with enough precision and image quality to be able to measure, in the image, a proper physical characteristic of the detectable marker, at the level of an individual droplet. In order to obtain usable images at the level of an individual droplet, it is indeed necessary to avoid having, in a given droplet, the grouped marked and magnetized combination and the cell aligned along the optical imaging axis A1. Indeed, in such a case, one would at least partially hide the other, which could lead to improper detection of the required physical property. Also, it has been experienced that proper focus was difficult to achieve, across one individual droplet and also between different cells in a given storage chamber. If any of the grouped marked and magnetized combinations and/or of the cell is out-of-focus in the image, the detection might be altered, especially when it involves evaluation or measurement of a physical characteristic in the image. This phenomenon can be at least in part attributed to the very shallow depth of field of the imaging devices, which are based on optical microscopes, used for imaging the droplets.

Thus, one aim of the invention is to provide an improved process and apparatus for analyzing biological cell activity which can be exploited in cell imaging field, where the optical images obtained for the detection step have a better focusing accuracy, allowing an assessment of both a physical property of the detectable marker and of the states of the cell(s) responsible for this recorded signal. One other aim of the invention is to provide an improved process and apparatus where the acquired optical images allow a follow-up in time of one or several individual encapsulated biological cells, with respect to their physiological state and at least a targeted secretion activity.

Another aim of the invention is to provide such an improved process and apparatus that could generate images that could be easily processed by an automated image processing software, and with which the relevant droplets for the study could be automatically identified, particularly thanks to the ascertainment that the individual encapsulated cells and the grouped marked and magnetized combinations are rightly located at their respective attributed locations inside the droplets.

The invention thus provides for a microfluidic droplet-based assay process for analyzing biological cell activity by detection of at least one target analyte contained in one or several droplets. The process comprises the step of creating several assay droplets of a liquid culture medium where an assay droplet contains:
paramagnetic particles;
detectable markers;
and, at least one biological cell potentially capable of releasing at least one target analyte;

wherein the paramagnetic particles and the detectable markers are able to bind with a target analyte to form a marked and magnetized combination with a target analyte.

The process includes storing several assay droplets along a single layer along a horizontal storage plane.

The process includes the generation of a magnetic field through the stored assay droplets for spatially arranging the paramagnetic particles inside the stored assay droplets.

The process includes a step of detecting, within a given stored assay droplet, a physical characteristic of the detectable markers contained in the given assay droplet, by optical imaging of the detectable markers.

The process is characterized in that the generation of a magnetic field generates a magnetic field such that the paramagnetic particles are attracted towards a lateral and inferior portion of the given stored assay droplet in which they are contained. In other words, the process is characterized in that the magnetic field induces the attraction of the paramagnetic particles towards a lateral and inferior portion of the given stored assay droplet in which they are contained. Such magnetic field is generated at least during said step of detecting.

According to optional features of the process, taken alone or in combination:
The magnetic field may be such that the paramagnetic particles are attracted towards a similarly disposed lateral and inferior portion of all the stored assay droplets.
The magnetic field may be asymmetric with respect to the stored assay droplets.
The process may comprise detecting fluorescence of fluorescent detectable markers and/or detecting radioisotope-labeled detectable markers.
The process may comprise providing, in the assay droplet, additional detectable markers able to bind with specific proteins expressed on the biological cell surface, to tag the biological cell.
The liquid culture medium may comprise at least one vital dye and/or at least one non-vital dye.

The invention also provides for an apparatus for a microfluidic droplet-based assay process for analyzing biological cell activity by detection of at least one target analyte contained in one or several droplets. Such apparatus comprises a storage chamber for storing several assay droplets of a liquid culture medium containing:
paramagnetic particles;
detectable markers;
and, at least one biological cell potentially capable of releasing at least one target analyte.

In such apparatus, the storage chamber is transparent in a detection process of the detectable markers and where the storage chamber is formed between two parallel top and bottom inner wall surfaces of the storage chamber, the inner wall surfaces extending parallel to a horizontal plane and being separated vertically by a height, such that the assay droplets are stored along a single layer.

The apparatus includes a magnetic field generator for spatially arranging the paramagnetic particles inside the assay droplets stored in the storage chamber.

The apparatus includes a detector of a physical characteristic of the detectable markers contained in a given assay droplet stored in the storage chamber.

The apparatus is characterized in that the magnetic field generator generates a magnetic field such that the paramagnetic particles are attracted horizontally towards at least one side of the storage chamber and vertically towards the bottom inner wall surface of the storage chamber. In other words, the magnetic field generator is configured to generate a magnetic field which attracts the paramagnetic particles horizontally towards at least one side of the storage chamber and vertically towards the bottom inner wall surface of the storage chamber.

According to optional features of the apparatus, taken alone or in combination:
The magnetic field generator may comprise one or several magnets forming a magnet assembly which is arranged below the level of the bottom inner wall surface of the storage chamber.
The inner bottom wall surface of the storage chamber may pertain to a bottom wall of the storage chamber which exhibits a bottom external wall surface opposite its inner wall surface, and magnet assembly may be arranged below the level of the bottom external wall surface.
The magnetic field generator may generate a magnetic field such that the paramagnetic particles are attracted horizontally towards a same side of the storage chamber.
The magnetic field generator may comprise one or several magnets forming a magnet assembly and having each magnetic poles, wherein at least one of the poles of each of the magnets is located horizontally to a same side of the storage chamber, which may be the side towards which the paramagnetic particles are attracted horizontally.
The magnetic field generator comprises one or several magnets forming a magnet assembly and having each magnetic poles, wherein both of the poles are located horizontally to a same side of the storage chamber, which may be the side towards which the paramagnetic particles are attracted horizontally.
The storage chamber may be elongated along a first direction of the horizontal plane, and in that both of the poles are located horizontally to a same side along a second direction of the storage chamber which is perpendicular to the first direction, where the same side may be the side towards which the paramagnetic particles are attracted horizontally.
The magnetic field generator may generate a magnetic field such that the paramagnetic particles are attracted, along the second direction, horizontally towards a side of the storage chamber.
The side of the storage chamber may be defined with respect to a centroid of the extension of the storage chamber along the horizontal plane.
The side of the storage chamber may be defined with respect to a periphery of the storage chamber along the horizontal plane.
The storage chamber may be part of a sample carrier device and the magnetic field generator may be affixed to said sample carrier device.
The magnetic field generator may be part of a detection station of the apparatus and the storage chamber may be part of a sample carrier device, the detection station and the sample carrier device being movable relative one to the other and being brought into coincidence for detection of at least one target analyte contained in one or several droplets contained in the storage chamber of the sample carrier device.

The invention also provides for a sample carrier which may comprise two plates forming the two walls of the storage chamber, in which the two plates may be affixed one to the other by an adhesive film sandwiched between the two plates, and wherein the thickness of the adhesive film determines vertically the height of the storage chamber. The adhesive film may comprise an internal cutout, the border of which delimits the extension of the storage chamber along the horizontal plane.

The processes and apparatus according to the invention will be further described in detail below with reference to the accompanying drawings showing some embodiments of the invention.

### In the figures:

FIG. 1 represents schematically a process for producing assay droplets which can be performed according to known technologies.

FIG. 2 represents an example of how a paramagnetic particle, a target analyte and a detectable marker can bind to form a marked and magnetized combination.

FIG. 3 represents schematically a detection station in an apparatus according to one possible embodiment of the invention.

FIG. 4 represents schematically a vertical transverse cross section of a storage chamber with a magnetic assembly according to one possible embodiment of the invention.

FIG. 5 represents a top view of the 4 main components of an embodiment of a sample carrier device comprising a storage chamber, before assembly.

FIG. 6 represents schematically a top view of sample carrier device according to FIG. 5.

FIGS. 7A, 7B represent schematically a top view of some elements in a droplet, in the absence of a target analyte, at two different stages of a process, and FIG. 7C represents a bright-field image of the droplet of FIG. 7B while FIG. 7D is a schematic representation of the droplet of FIG. 7B, visualized under a wavelength appropriate to excite the detectable markers of the droplet.

FIGS. 8A, 8B represent schematically a top view of some elements in a droplet, in the presence of a target analyte, at two different stages of a process, and FIG. 8C represents a bright-field photography of the same droplet as in FIG. 8B while FIG. 8D is a schematic representation of the droplet of FIG. 8B, visualized under a wave length appropriate to excite the detectable markers of the droplet.

FIGS. 9A and 9B represent a top view and cross section of a variant of the positioning of a magnet assembly with respect to a storage chamber, according to another embodiment of the invention.

FIGS. 10A and 10B, 11A and 11B, 12A and 12B, are similar view representing further embodiments of the invention.

FIG. 13 is an image of several droplets in a storage chamber, that can be obtained when the applied magnetic field is oriented as in the case of a magnetic assembly arranged as in FIG. 4 and FIGS. 10A and 10B, 11A and 11B.

FIG. 14 is an image of several droplets in a storage chamber, that can be obtained when the applied magnetic field is oriented as in the case of a magnetic assembly arranged as in FIGS. 9A and 9B, 12A and 12B.

The invention provides for a microfluidic droplet-based assay process for analyzing biological cell activity by detection of at least one target analyte contained in one or several droplets. It also provides for an apparatus for implementing such a process.

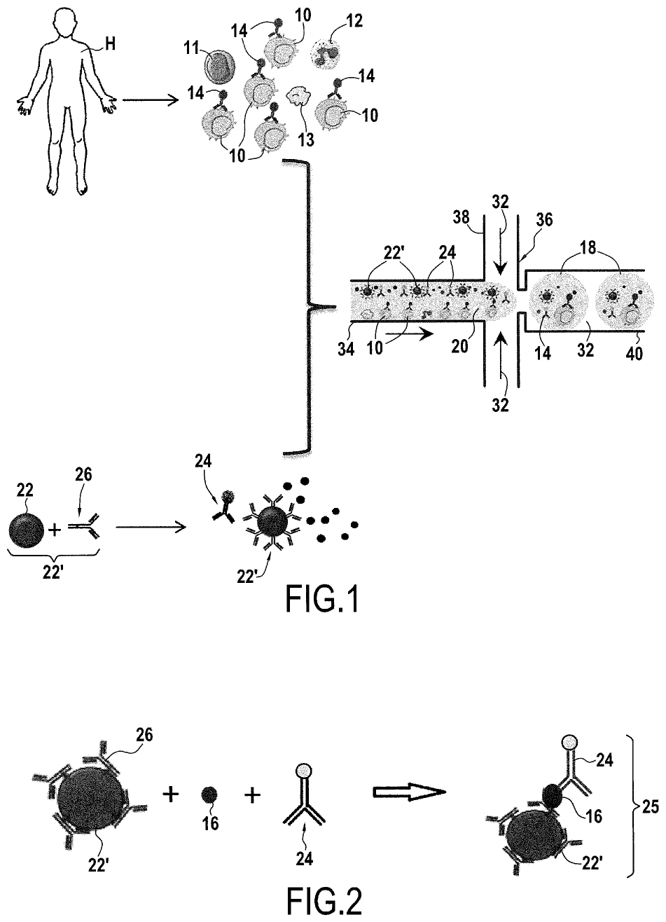

The apparatus and the process may be used for analyzing the activity of any type of biological cell, including eukaryotic cells (animal, plant, fungal and protozoan cells) or prokaryotic cells. In FIG. 1 is represented schematically the extraction and preparation of biological cells 10, 11, 12, for example from a living human H. The extraction may include live cells 10, and may include dead cells 13. In the exemplary application which will be referred hereafter, the biological cells comprised human monocytes 10 which were enriched using an isolation kit (for example as provided by StemCell™ Technologies, 40 Rue des Berges, Miniparc Polytec, Bâtiment Sirocco, 38000 Grenoble, France). Human monocytes can be specifically tagged by anti-CD14 fluorescent antibodies 14 to distinguish them from other cell-types 11, 12.

It is here contemplated that, under certain circumstances, such cells might have a biological activity whereby they are able to produce, directly or indirectly, at least one certain species of analyte 16 (shown in FIG. 2), which will be detectable by a detection process.

In the process and apparatus according to the invention, it will be sought to detect such analyte which will hereinafter be called a target analyte. It will be possible to devise a process and an apparatus according to the invention where several analytes, by the meaning of different species of analytes, may be targeted, thus forming different target analytes. Such different target analytes could be the result, direct or indirect, of the same biological process inside the biological cell 10, or of different biological processes inside the same biological cell 10.

A target analyte 16 can be for example any protein that may be released by a biological cell, such as cytokines (e.g. chemokines, interferons, interleukins, lymphokines, tumour necrosis factors, growth factors), antibodies, lectins, hemoglobin, hormones, toxins. A target analyte 16 may also microRNAs (miRNAs), which have recently been disclosed as being possible circulating chemical mediators that would be involved in a particular form of intercellular communication. Therefore, such secreted miRNAs may also be contemplated as target analytes within the meaning of the invention. In the exemplary application, human monocytes 10 were studied for their aptitude to secrete TNF-alpha 16, after stimulation by lipopolysaccharide (LPS) used as an active agent.

As schematically shown in FIG. 1, the process comprises the steps of creating several assay droplets 18 of a suspension fluid, typically an aqueous liquid 20, more typically a liquid culture medium 20, where an assay droplet contains
paramagnetic particles 22, 22′;
detectable markers 24;
and, as discussed above, at least one biological cell 10 potentially capable of releasing at least one target analyte.

In the exemplary application, said suspension fluid was a cell culture medium ensuring cell viability throughout the course of the experiment. By “culture medium”, it is meant a composition comprising nutritive elements necessary for the expression of a metabolism and/or the growth of the studied biological cell. Said culture medium is liquid, more particularly an aqueous liquid culture medium adapted to cell survival, at least during the assay. In addition to water, an aqueous culture medium generally comprises one or several of:
a source of carbon and of energy (generally glucose, but according to the nature of the biological cell, saccharides such as lactose, maltose, etc. may be appropriate)
a source of calcium (for example CaCl₂);
a source of nitrogen and sulfur (for example (NH₄)₂SO₄);
a source of magnesium (for example MgCl₂);
trace elements (for example, salts of Cu, Zn, Co, Ni, B, and/or Ti).

Optionally, a culture medium may also comprise a pH buffer and/or various salts to maintain the medium at an appropriate pH and/or an appropriate osmolality.

Also, as will be detailed below, the suspension fluid may comprise, for example as part of the culture medium, at least one vital dye and/or at least one non-vital dye.

Additionally, the suspension fluid may contain, for example as part of the culture medium, at least one active agent potentially capable of interfering (stimulating, increasing, or inhibiting) with the release of said at least one target analyte 16 by the biological cell. The active agent may be a known activator/stimulator used to amplify and/or accelerate the secretion of the target analyte, with a view to improving the sensitivity of the assay and/or obtaining more accurate results and/or obtaining results more quickly. The active agent can also be a molecule that is tested (for example a drug candidate) for its ability to block the secretion of the target analyte or on the contrary to induce and/or enhance the secretion of this target analyte.

As shown in FIG. 2, the paramagnetic particles 22, 22′ and the detectable markers 24 contained in an assay droplet 18 are able to bind with a target analyte 16 to form a marked and magnetized combination 25 with a target analyte 16.

The paramagnetic particles 22 are attracted by an externally applied magnetic field. On the other hand, the paramagnetic particles 22 do not retain any magnetization in the absence of an externally applied magnetic field. Paramagnetic particles 22 may comprise paramagnetic beads. The paramagnetic particles 22 may comprise nanoparticles, which may typically exhibit a largest dimension comprised between 10 and 1000 nanometers, preferably between 100 and 500 nanometers. Typically, the paramagnetic particles 22 can be spherical beads, with a diameter of about 300 nanometers, i.e. comprised between 200 and 400 nanometers.

The paramagnetic particles 22 are preferably monodisperse, by exhibiting, within a given population, a maximum variation of the largest dimension of the particles which is less than preferably 20%, more preferably less than 10%, of the largest dimension of all paramagnetic particles in the population. Paramagnetic nanoparticles are well suited to immunodiagnostic technologies owing to their large surface-to-volume ratio, their monodispersity, their stability and easy manipulation in a magnetic field. They provide an important toolbox for chemical modification and functionalization of their outer surface. Moreover, some evidence showed that nanoparticle-based biosensors exhibit better homogeneity and sensitivity than microbead-based biosensors where the beads have a dimension larger than 1000 nanometers. The paramagnetic particles 22 may exhibit carboxyl (COON) or amine (NH₂) groups on their outer surface, for facilitating the coupling of capture elements.

In the exemplary application, the used paramagnetic particles 22 were Carboxyl-Adembeads 0213 from Ademtech SA—Bioparc BioGalien—27, allée Charles Darwin—33600 PESSAC—France, as described in their protocol brochure 0213 v2.6.

In order to be able to bind specifically with a target analyte 16, the paramagnetic particles 22 need to be functionalized. Functionalization of the paramagnetic particles comprises coupling, to said outer surface of the paramagnetic particles, capture elements 26 which are capable of binding with the target analytes 16. Such capture elements 26 may be antibodies directed against target analytes 16, for example G type immunoglobulin (IgG), monoclonal or polyclonal, directed against target analytes 16. In the exemplary application, capture elements 26 were monoclonal anti-TNF-alpha IgG (ref. 3510-5-250, MABTECH, Sweden).

In the last decade, several antibody coupling procedures have been developed which can be implemented for coupling antibodies to the paramagnetic particles. In contrast to random coupling, it is preferred to implement procedures enabling an oriented coupling of the antibodies, such as IgG 26, onto the paramagnetic particles, so that the antigen-binding (Fab) fragment recognition sites of the antibody remain exposed. This improves the analyte detection and biosensor sensitivity. It is for example possible to use a boronic acid (BA) based coupling procedure for precise orientated coupling of IgGs on the surface of paramagnetic nanoparticles 22 (Duval F., van Beek T. A., Zuilhof, H. “Key steps towards the oriented immobilization of antibodies using boronic acids”; published in The Analyst, 140, 6467-6472 (2015)). BA molecules may be thus coupled on the surface of carboxylated beads to act as an affinity head-group for covalent and site-specific IgG attachment. The BA-based procedure maintains the integrity of IgG structure and immunoactivity. BA forms a cyclic boronate diester with the diols of glycan chains located on IgG crystallizable fragment Fc domain 30, far away from the active binding site. The procedure does not require modification of IgGs, thus preserving the biological activity of antibodies. There exists a large range of commercially available BA from different suppliers. Water-soluble BA molecules, reference A71751 from Merck (formerly, Sigma-Aldrich), a 3-aminophenylboronic acid hemisulfate salt, CAS Number 66472-86-4, have proven to provide optimal results.

Alternatively to antibodies, capture elements 26 may consist in entities known as natural or synthetic/mimetic of receptors or of ligands of said target analytes 16.

Detectable markers 24 are chosen for their ability to combine with the target analyte 16.

A detectable marker 24 may be an antibody, monoclonal or polyclonal, directed against target analytes 16, and may have at least one physical characteristic which may be detected in a detection process. In this regard, an antibody used as a detectable marker in the sense of the invention is advantageously labelled with fluorescent dye or a radioisotope.

A detectable marker in the sense of the invention may be a fluorescent-labelled marker and/or a radioisotope-labelled marker that is able to bind with a target analyte.

In the exemplary application, the detectable markers 24 were second monoclonal anti-TNF-alpha IgG. Those second monoclonal anti-TNF-alpha IgG were different from the first monoclonal anti-TNF-alpha IgG used as capture elements 26 in that they bind to another part of the target analyte 16. Additionally, they were coupled to phycoerythrin (PE), a fluorescent dye. Typically, in the exemplary application, the detectable markers 24 were PE-anti-TNF-alpha IgG (ref. 120-014-229, MILTENYI BIOTEC, Germany).

Alternatively to antibodies, detectable markers 24 may consist in entities known as natural or synthetic/mimetic receptors said target analytes 16 or of ligands of, possibly labelled with a fluorescent dye or a radioisotope.

Obviously, capture element 26 and detectable marker 24 are chosen such that they can bind simultaneously to a same target analyte 16.

In the event that the detection of several target analytes is desired, then different detectable markers 24 labelled with different labels (such as different fluorescent dyes or radioisotopes) may be used; each of these detectable markers with its specific fluorescent dye or radioisotope being associated to a specific target analyte.

In addition to the detectable markers which are able to bind with the target analyte, the process may comprise providing, in the assay droplet, additional detectable markers which able to bind with the biological cell 10. Such cell-directed additional detectable markers, which may also be fluorescent or radioisotope-labeled, are advantageously used in order to identify the cell-type, and confirm that the encapsulated cell fits with the cells to study. For example, detectable markers directed against CD14 and/or CD33 allow the identification of macrophages and monocytes, whereas detectable markers directed against CD3 and/or CD4 and/or CD8 allow the identification of T-cells. Of course, the cell-directed additional detectable markers should be detectable separately from the detectable markers which are able to bind with the target analyte. For example, they should exhibit a different fluorescence wavelength.

Regarding the paramagnetic particles, paramagnetic particles of a same type may be used, the surface of which is coated with different capture elements. The set of these capture elements at the surface of each paramagnetic particle, allows a binding with all the target analytes. According to another embodiment, a mixture of paramagnetic particles of several types may be used. The surface of each paramagnetic particle may be coated with a specific capture element capable of binding with a specific target analyte. The set of paramagnetic particles used in such a case preferably allow binding with all the target analytes.

As seen in FIG. 1, the functionalized paramagnetic particles 22′, detectable markers 24 and biological cells are mixed in a suspension fluid 20, preferably a liquid, typically an aqueous liquid, more typically a liquid culture medium. From this suspension liquid, droplets 18 are formed.

Jöensson et al, 2012 summarizes various techniques for producing droplets according to the invention. Eyer et al., 2017 discloses more precisely such a method. Preferably the droplets are produced as an emulsion of droplets of the suspension fluid, typically an aqueous liquid culture medium 20, in a dispersion medium, typically an oil-based fluid 32, preferably as a homogenous monodisperse emulsion of aqueous liquid droplets 18 in an oil-based liquid 32.

In the invention, an assay droplet has a volume which is typically below 200 picoliters (pL). Typically, the preferred volume for an assay droplet 18 is comprised between 4 pL and 600 pL, preferably within the range from 30 pL to 100 pL. In the exemplary application, the volume of the assay droplets 18 was about 50 picoliters.

This step of droplet formation, where assay droplets 18 are formed from the suspension fluid containing paramagnetic particles 22, 22′, detectable markers 24, and at least one biological cell 10, is often called encapsulation, because the paramagnetic particles, detectable markers, and biological cell(s) are encapsulated inside the assay droplet 18.

As shown very schematically in FIG. 1, encapsulation may involve flowing a solution of the suspension fluid, incorporating the functionalized paramagnetic particles 22′, detectable markers 24 and biological cells, in a microchannel, for example capillary tube 34, towards a cross-junction 36 with another microchannel, for example a capillary tube 38 in which circulates a flow of the dispersion medium 38. By proper calibration, an emulsion of monodisperse droplets of the suspension fluid in the dispersion fluid is collected in a downstream output capillary tube 40.

Such microfluidic droplet formation techniques are known in the art. The fabrication and operation of a microfluidic chip for droplet production has been previously described in Mazutis et a., 2013. The microfluidic system has been optimized to co-encapsulate a single mammalian cell and reagents in one droplet. The device may in fact contain three inlets for bringing respectively (I) the dispersion fluid, (II) the suspension of cells in the suspension fluid and (III) the assay reagents, which may include functionalized paramagnetic particles, detectable markers, and possibly other reagents, such as live/dead cell probes, stimulating agents, etc. . . . The device has, as described above, at least one outlet for collecting the produced droplets.

In the devices as described above, assay droplets are formed at the flow-focusing junction 36. Preferably the device and the flows in the device are calibrated so the flows at the junction 36 are laminar.

In order to prevent clogging of the microfluidic device during experiments, passive filters may be added at one or several of the inlets, or at each inlet. Fluid resistors and/or serpentine channels may be used to damp fluctuations of the flow within the microchannels. Such a droplet generating device produces highly monodisperse and stable aqueous droplets in inert fluorinated oil over extended periods of time.

The process may be devised such that an assay droplet 18 comprises other reagents. For example, assay droplets may include an active agent in the form of a stimulation agent for stimulating the activity of the cell, preferably stimulating especially the biological process from which the target analyte originates. In the exemplary application, where the bioassay is devised to monitor TNF-alpha secretion by a single monocyte, the stimulation agent was lipopolysaccharide (LPS). Using a three inlet device as described just above, the contact between cell and assay reagents occurs only after encapsulation in the assay droplet 18. Therefore, behavior of cell dynamics is observed only after stimulation within the assay droplet 18.

In order to achieve monodispersity of the functionalized paramagnetic particles it can be necessary to prevent them from aggregating. One way to prevent this can include sonicating the functionalized paramagnetic particles 22′ for 1 to 3 minutes in a water bath sonicator prior to encapsulation. Together with the use of nanoparticles as paramagnetic particles, this has proven to notably improve the performance of the process, as each individual cell is encapsulated within an assay droplet with the same amount of functionalized paramagnetic particles, and the formation of droplets containing a cell and no functionalized paramagnetic particles is avoided.

As in the know prior art, the process includes storing several assay droplets 18 along a single layer along a horizontal storage plane, here in a storage chamber 42.

In the shown embodiment, the storage chamber 42 is formed in a sample carrier device 44.

In the example, depicted in FIGS. 4, 5 and 6, the storage chamber 42 is designed to be transparent in a detection process of the detectable markers 24. In the example, the detection process is an optical detection process.

Therefore, the storage chamber 42, and in the example, the part of the sample carrier device 44 which forms the storage chamber 42, is optically transparent, at least at the wavelengths involved in the detection process, which may include wavelengths in the visible domain, and/or wavelengths in the infrared domain and/or in the ultraviolet domain.

In the shown example, the sample carrier device 44 comprises two plates, namely a top plate 46 and a bottom plate 48, which extend parallel to each other. The plates 46, 48 can be made of glass, or of similar material, for example a transparent polymer material. In operation of the apparatus, at least during the step of detection of the detectable markers which will be discussed below, the two glass plates 46, 48 are horizontal. They form the two top and bottom walls of the storage chamber 42. In the exemplary application, the plates were plates of 75 millimeters long by 25 millimeters large.

In the exemplary application, the two glass plates 46, 48 are affixed one to the other by an adhesive film 50, preferably having adhesive on both sides, which is sandwiched between the two glass plates. The thickness of the adhesive film 50 determines vertically the height of the storage chamber. Indeed, the storage chamber 42 is formed between two parallel top 47 and bottom 19 inner wall surfaces, which are respectively the bottom surface of the top plate 46 and the top surface of the bottom plate 48. The inner wall surfaces 47, 49 extend, in operation of the apparatus, at least during the detection phase of the process, parallel to a horizontal plane and, they are separated vertically by a height, which is the height of the storage chamber 42.

The height of the storage chamber 42 is designed to make sure that the stored assay droplets are stored along a single layer that layer extending along a horizontal plane, here parallel to the plates 46, 48. Thus the height of the storage chamber is designed according to the volume of assay droplets 18. The height of the storage chamber 42 is preferably less than 2 times, preferably less than 1.5 times the diameter of a sphere having the same volume as the assay droplets 18. On the other hand, the height of the storage chamber is preferably more than 0.5 times, preferably more than 0.8 times the diameter of a sphere having the same volume as the assay droplets 18. Typically, for a volume of the assay droplets comprised between 4 picoliters (pL) and 600 picoliters (pL), the height of the storage chamber may be between 10 micrometers (μm) and 200 micrometers (μm).

As visible in FIG. 5, the adhesive film 50 comprises an internal cutout 52, the border 54 of which delimits the extension of the storage chamber along the horizontal plane. In the exemplary application, an about 40-50 micrometers thick double-sided adhesive film 50 was used to bind the two plates 46, 48 together, by applying moderate pressure with clamps, followed by 1-hour incubation at 90° C. Studies have demonstrated that the bond strengths obtained with double-sided adhesive film is reproducible and comparable to that of plasma and corona discharge. Several double-sided adhesive films with different thickness are commercially available (for example from 3M®) and any shape and size of the storage chamber can be obtained by cutting the internal cutout 52 at a desired shape and size in the film with a simple knife plotter or with a laser cutting machine. Adhesive films have a strong adhesion to glass surfaces and are stable at high temperature, thus preventing fluid leakage and water loss from the emulsion. Polyester double-sided adhesive films have proven to provide robust assembly of glass plates and accurate control of height of the storage chamber 42.

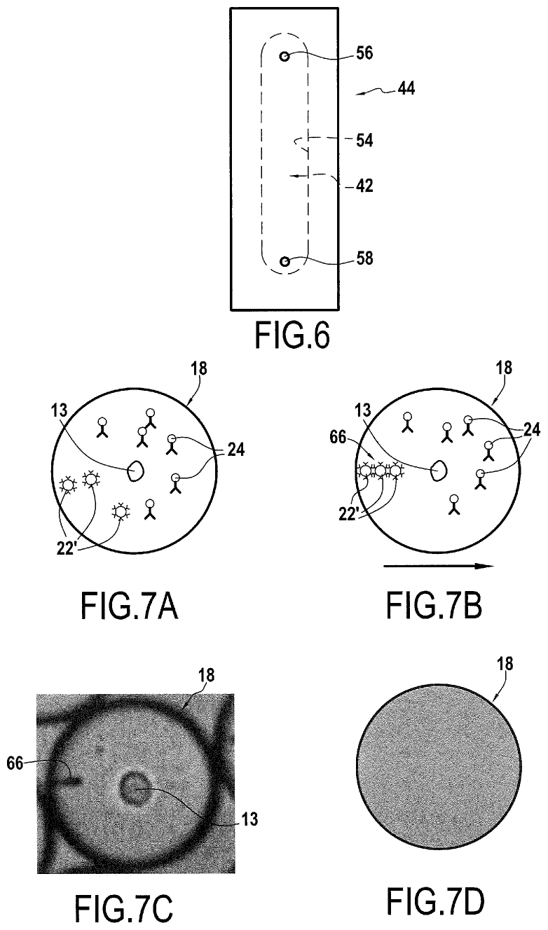

Also shown on FIGS. 5 and 6 is the presence of at least one inlet port for the storage chamber. In the shown embodiment, the sample carrier device 44 comprises two ports, an inlet port and a vent port, which both give access to the storage chamber 42. FIGS. 5 and 6 show two holes 56, 58 made in one the plates, here the top plate 46, in correspondence with the internal cutout 52, so that the holes provide access to the storage chamber 42. One hole 56 may be used as an inlet port, while the other hole 58 may serve as a venting port, to ease the filling of the storage chamber by allowing trapped air, or excess of suspension, to escape from the storage chamber 42 when a suspension containing assay droplets is introduced through the inlet port. The ports are preferably equipped with connectors on the external side (not shown in the Figures).

The process includes detecting, within a given stored assay droplet, a physical characteristic of the detectable markers contained in the given assay droplet, by optical imaging of the detectable markers. This step is referred to as the detection phase of the method. This step of detecting, and the detection as a whole may include, or may consist in, a mere analysis of presence or absence of a given physical characteristic in an image, or an analysis of its presence in an image above and/or below a threshold, which may be predetermined or not; or within a range, predefined or not. The steps of detecting may comprise the evaluation of the value of the physical parameter, including in view of dosing the physical characteristic, and thus of dosing the underlying quantity of target analyte.

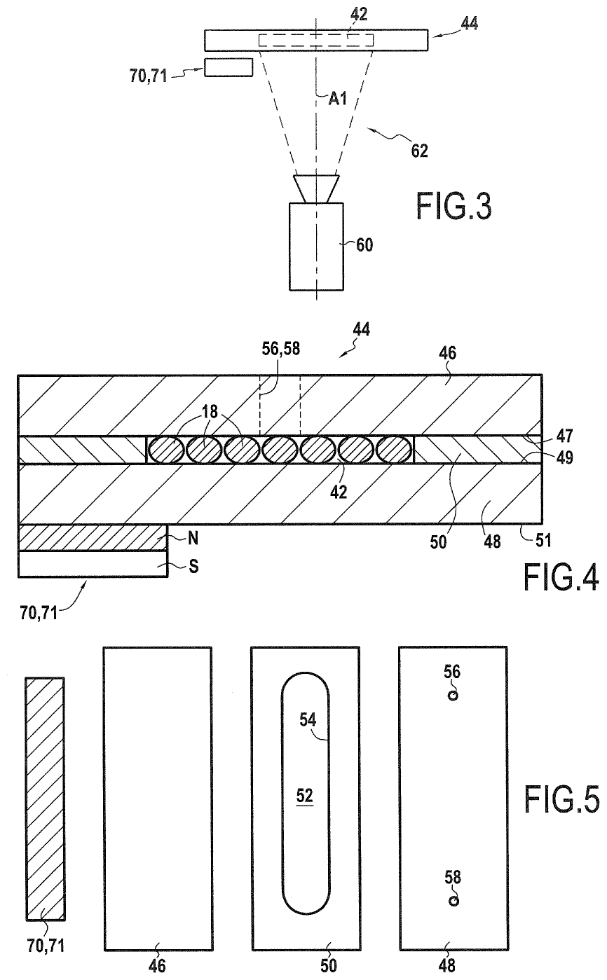

To that effect, the apparatus shown in FIG. 3 includes a detector 60 of a physical characteristic of the detectable markers 24 contained in a given assay droplet 18 stored in the storage chamber 42. The detector 60 may be part of a detection station 62 of the apparatus. Typically, the detector 60 comprises at least one camera which is able to take pictures of the assay droplets 18 stored in the storage chamber 42. The camera views the storage chamber 42 along an optical imaging axis A1 which is perpendicular to the horizontal plane, thus vertical. Optical imaging may be performed from below the storage chamber 42, i.e. through its bottom plate 48. Of course, the detector 60 may comprise lenses and/or mirrors such that the camera itself may be arranged with a different orientation, not necessarily below the storage chamber 42 at the detection station 62. The detection station comprises several lights for illuminating the storage chamber. The lights may have different illumination wavelengths, with for example one or several lights having a monochromatic illumination wavelength, and/or one or several lights having a limited illumination wavelength bandwidth, for example limited to a part of the ultraviolet spectrum or to a part of the infrared spectrum, and/or one or several lights having a broad illumination wavelength bandwidth, for example covering the visible spectrum or part thereof to achieve a white light illumination. The lights may be selectively activated to achieve different illumination conditions. Among these lights that may be selectively activated, some are able to stimulate the detectable markers and, optionally, the vital dye(s) and/or the non-vital dye(s) that are used to carry out the process according to the invention.

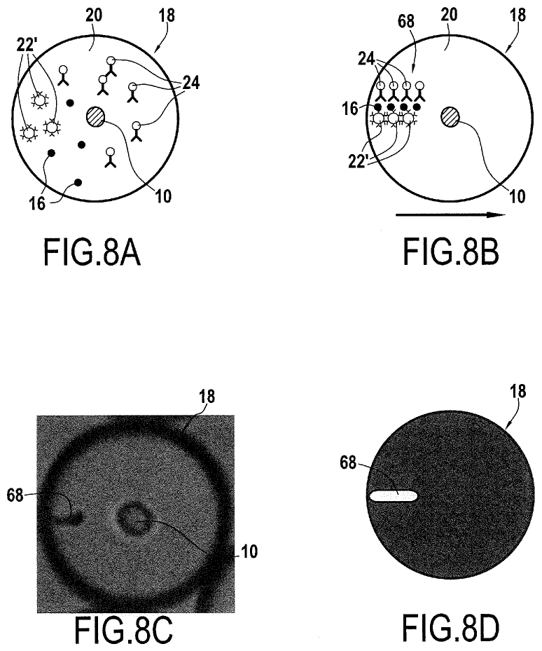

The camera may typically include an imaging array sensor, such as a CCD array sensor or a CMOS array sensor where the imaging array sensor comprises an array of sensor element. The camera operates an optical imaging process by forming an optical image of the storage chamber on the imaging array sensor. The imaging array sensor delivers an image of the storage chamber where the detectable markers may be visible, under certain conditions which are explained below in relation to FIGS. 7A to 7D and 8A to 8D. The image can be a digital signal. As part of the detection phase, the image can be analyzed by a human operator or by an automated process, such as a computer image analysis process. Such analysis may involve identifying and counting the number of cells, aggregates, etc. . . . , in one or several images of the storage chamber, and may involve comparing such data between images of the same assay under different illumination conditions.

In an exemplary application, the detector comprises a monochromatic camera which delivers a greyscale digital image of its field of view. In the image delivered by the monochromatic camera one point of an object in the camera's focusing plane corresponds to one single point in the image, where the single point has a grey level representative of the intensity of the light received by the corresponding sensor element.

In some embodiments, the detection phase involves detecting fluorescence of fluorescent detectable markers. With a fluorescent detectable marker, the physical characteristic of the detectable markers which is detected by the detector is the fluorescence light emitted by the marker when it is properly excited. To that effect, the detector, typically the imaging array sensor discussed above, must be sensitive in a wavelengths bandwidth containing the fluorescence wavelength(s) of the detectable marker. The detector may comprise one or several optical bandwidth filter(s) which can be selectively activated and deactivated in accordance with the detectable marker(s) and, optionally, with the vital dye(s) and/or the non-vital dye(s) that are used to carry out the process according to the present invention, for optically imaging the storage chamber. Different optical filters may therefore be used, for the acquisition of different images, for detecting different markers, optionally, different vital or non-vital dyes emitting different fluorescence wavelengths.

The detection phase may also comprise acquiring, with the detector 60, a bright-field image.

In some embodiments, the detector 60 can perform imaging of the entire storage chamber 42 in one single frame, corresponding to the field of view of the detector or to part of it. However, the field of view of the detector 60 may be smaller than the extension of the storage chamber 42, or smaller than a region of interest of the storage chamber. In such a case, the process and apparatus may be devised such that several juxtaposed frames are taken, each frame being an image of a portion of the region of interest, with a relative displacement between the storage chamber and the detector between each frame.

In the exemplary application aiming at tracking the behavior of cells when trapped within assay droplets, a region of interest having an overall square area of 220 square millimeters of a monolayer array of such assay droplets, immobilized in the storage chamber, was imaged by the detector. In the experimental setup for the exemplary application, the sample carrier device 44 having the storage chamber 42 was placed on the motorized stage of an inverted epifluorescence microscope (Nikon®). Both the microscope and the sample carrier device 44 having the storage chamber 42 were encompassed in a box at 37° C. A 10× magnification objective (CFI Plan Apochromat Lambda 10×, Nikon, N.A.=0.45, working distance 4 mm) was used to image 125 joint frames, of 2048×2048 pixels, in 5 different imaging channels, corresponding to 5 different imaging wavelength bandwidths. Focus was manually set on the first field so that the contours of droplets and the paramagnetic particles appear as dark objects on a clear background in a bright-field image.

The magnification used for image acquisition during the detection phase can also be a limiting factor of the procedure. For example, a 10× magnification can be a compromise between scanning time and resolution, as it enabled imaging simultaneously about 600 droplets per frame with a sufficient resolution so that all the relevant objects within the image (i.e. droplets and encapsulated cells and marked and magnetized combinations) could be discriminated during an automated image processing.

As both automated object detection and fluorescence intensity measurements rely on focus quality, control of focus drift throughout the imaging procedure (of a large area and over a long duration) is crucial. Mechanical effects, vibrations, thermal changes and droplet movement can cause the focus to drift. Droplet coalescence is also a factor that can affect focal plane of the droplet array. This limitation may be addressed by using manual focus or automatic focus. However, this limitation was preferably addressed in the exemplary application using the Perfect Focus System (PFS) of the Nikon® microscope to provide fast and efficient real-time adjustment of the focal plane during the long-lasting acquisitions and surface scanning.

However, such focusing may not be helpful if different elements in a same picture frame are not in the same focal plane.

The known process includes the generation of a magnetic field through the stored assay droplets for spatially arranging the paramagnetic particles inside the stored assay droplets. Therefore the apparatus includes a magnetic field generator creating a magnetic field across the storage chamber, for spatially arranging the paramagnetic particles inside the assay droplets stored in the storage chamber. Indeed, by providing such a magnetic field, all the paramagnetic particles contained in one assay droplet are thus gathered in one region of the droplet which depends on the orientation of the magnetic field at the location of the droplet.

The effect of the magnetic field is exemplified in schematics shown respectively in FIGS. 7A, 7B, 7C, 7D and 8A, 8B, 8C, 8D. FIGS. 7A and 8A are a representation of an assay droplet 18 containing functionalized paramagnetic particles 22′ and detectable markers 24, in the absence of any magnetic field. All these elements are thus randomly dispersed within the droplet 18. In FIG. 7A, no target analyte is present, whereas the case of presence of target analytes corresponds to FIG. 8A. In reality, the presence of target analytes, which are supposed to be produced by a non-shown biological cell, would lead to the formation of marked and magnetized combinations, as shown in FIG. 8B.

FIG. 7B and FIG. 8B depict the assay droplets 18 in presence of a magnetic field according to the invention. Due to this magnetic field, the paramagnetic particles 22′ are gathered in one region of the assay droplet 18, forming an aggregate 66, 68 of paramagnetic particles 22′. In the schematics, the paramagnetic particles are shown to be arranged, under the influence of the externally applied magnetic field, in a rod-like arrangement, but other shapes are possible for the aggregate 66, 68. However, in FIG. 7A is shown that, in the absence of a target analyte, the detectable markers do not bind to the functionalized paramagnetic particles 22′, or at least not to any significant level. Thus, even though the paramagnetic particles 22′ are aggregated in one region of the droplet, the detectable markers 24 remain randomly dispersed and not show any relocation of the detectable marker on the aggregated paramagnetic particles. In FIG. 7B, without the presence of target analytes in the assay droplet, it is shown that an aggregate 66 of functionalized paramagnetic particles 22′ may form, but such aggregate 66 does not contain detectable markers, at least not in any significant quantity, and can thus be called a non-marked aggregate 66.

In FIG. 7C is shown a bright-field image that can be obtained with the process according to the invention. As in this example shown in FIG. 7C, it is possible that paramagnetic particles are visible in a bright-field image, thanks to the size of the individual paramagnetic particles and/or to the size of the aggregate 66 formed when under the influence of the externally applied magnetic field. As shown schematically on FIG. 7D, under an excitation light of a specific wavelength able to stimulate the detectable markers 24, the droplet 18 remains uniformly and weakly fluorescent, because the detectable markers, now fluorescent, remain disseminated/dispersed in the entire droplet. The non-marked aggregate 66 is not visible under such lighting.

To the contrary, FIG. 8B illustrates that, in the presence of target analytes, which have caused the formation of marked and magnetized combinations 25, the aggregate 68 formed by the paramagnetic particles under the influence of the magnetic field causes an aggregation, in the same aggregate 68, also of detectable markers 24. Such an aggregate can be called a marked aggregate 68. As illustrated in FIG. 8C, such marked aggregate 68 would possibly be visible in a bright-field image, similarly to the case of FIG. 7C, but more importantly now the detector 60 is capable of imaging, as illustrated in FIG. 8D the presence of the detectable markers in the marked aggregate, thanks to the aggregation, thus to the localized higher concentration of the detectable markers. When submitted to an excitation light of a specific wavelength able to stimulate the detectable markers 24, the droplet 18 appears with a fluorescent-marked aggregate 68. Indeed, the detectable markers, fluorescent under the excitation, are visibly relocated and concentrate on the aggregate 68, and the rest of the droplet 18 appears darker.

However, according to the invention, the process and apparatus are devised, i.e. configured, in a way that the magnetic field, which is generated across the storage chamber, and thus across the assay droplets contained in the storage chamber, is, at least during the detection phase, such that the paramagnetic particles are attracted towards a lateral and inferior portion of the given stored assay droplet in which they are contained. This is achieved thanks to the magnetic field generator which generates a magnetic field such that the paramagnetic particles are attracted horizontally towards at least one side of the storage chamber and vertically towards the bottom inner wall surface 49 of the storage chamber 42. The terms “inferior”, “bottom” and “below” are used herein with reference to the orientation of gravity, the orientation of which also determines the horizontal plane directions.

Indeed, tests have shown that, during the detection phase, a biological cell contained in a given assay droplet 18 tends to be located at the center of the droplet, when viewed along a vertical optical viewing axis, and towards the bottom of the droplet, by the mere action of gravity on the cell and of the shape of the droplet.

To the contrary, thanks to the properly arranged magnetic field, the aggregate, which may contain detectable markers bound to the paramagnetic particles, is attracted horizontally towards the side of the given droplet. Thus, by being to the side, the aggregate of detectable markers is, with respect to the optical viewing axis, laterally offset in a horizontal plane from the biological cell contained in the given droplet. This avoids or limits the risk that the aggregate and the cell are wholly or partially hidden of view one by the other along the vertical optical imaging axis Al.

Moreover by generating a magnetic field which attracts the paramagnetic particles towards the bottom of the storage chamber, thus towards the bottom of the assay droplet 18, it is obtained that the paramagnetic particles, including in the form of a marked aggregate 68, are located, along the optical viewing axis A1, in a same horizontal plane as the biological cell 10 contained in the same droplet, or with at least with a very little distance along the optical viewing axis. This ensures that, despite a possible very shallow depth of field, an image of the assay droplet 18 may show both the biological cell 10 and the paramagnetic particles, in the form of a marked aggregate 68, with comparable sharp focus. Such sharp focus of both elements will greatly facilitate identification of both elements, including by automatic computerized shape recognition, and will in parallel allow an accurate of determination of the physical characteristic which is to be detected from the detectable markers.

Therefore, the paramagnetic particles are attracted towards a lateral and inferior portion of the assay droplet, and finally find themselves located side by side with the cell which has fallen to the bottom of the assay droplet, under a phenomenon of sedimentation.

In reference with FIG. 7C and FIG. 7D, the absence of TNF-alpha secretion is marked by an absence of relocation and concentration of the fluorescence. In the assay droplet, the non-marked aggregate 66 and the biological cell 13 are located side by side, and both appear clearly on the bright-field image. The absence of TNF-alpha secretion is thus not due to a lack of cell encapsulation. An additional use of a vital dye and/or a non-vital dye would make it possible to check the cell viability.

In this regard, fluorogenic mitochondrial stains such as the ones belonging to the MytoView™ range are examples of vital dyes particularly useful to label live cells. CellTiter-Blue® Cell Viability Assay, from Promega Corporation, 2800 Woods Hollow Road, Madison, Wis. 53711-5399 USA may be used also to label live cells. On the other hand, cell membrane-permeable fluorogenic caspase substrates such as the ones belonging to the NucView® range allow a detection of apoptosis and are therefore particularly interesting examples of non-vital dyes to label dead cells. MultiTox-Fluor Multiplex Cytotoxicity Assay, from Promega Corporation, may be used to measure the number of live and dead cells simultaneously. HCS DNA damage Kit (H10292), from Thermo Fisher Scientific Inc., may also be used.

In reference with FIG. 8C and FIG. 8D, a relocation and concentration of the fluorescence are observed. A secretion of TNF-alpha is detected. In the assay droplet, the marked aggregate 68 and the biological cell are located side by side; they both appear clearly on the bright-field image. This allows to check that only one cell has been encapsulated in the assay droplet, and the recorded fluorescence intensity that is correlated with the quantity of secreted TNF-alpha is actually linked to the activity of a single cell. An additional use of a vital dye and/or a non-vital dye would make it possible to check the cell viability.

In some cases, several cells can be encapsulated in one same assay droplet. The process and apparatus according to the invention allow a following up of each assay droplet, in time. Therefore, it can be checked out whether several cells were co-encapsulated from the beginning at the encapsulation stage, or do come from a cell division occurring during the assay.

In some embodiments, the magnetic field is such that the paramagnetic particles are attracted towards a similarly disposed lateral and inferior portion of all the stored assay droplets. This is typically achieved with magnetic field generator which generates a magnetic field such that the paramagnetic particles are attracted horizontally towards a same side of the storage chamber.

Typically, such magnetic field can be asymmetric with respect to the stored assay droplets. This may be achieved by using an apparatus in which the magnetic field generator comprises one or several magnets, especially one or several permanent magnets or electromagnets, forming a magnet assembly 70 which is arranged horizontally asymmetrically with respect to the storage chamber, and below the level of the storage chamber 42.

It can be noted that, preferably, optical imaging may be performed from the side of the storage chamber 42 along the vertical axis towards which the aggregate is attracted by the magnetic field, thus for example from below the storage chamber 42, i.e. through its bottom plate 48. Indeed, because the cells tend to be attracted towards the bottom plate, this allows minimizing the thickness of any suspension fluid or dispersion fluid in the light path for the imaging process.

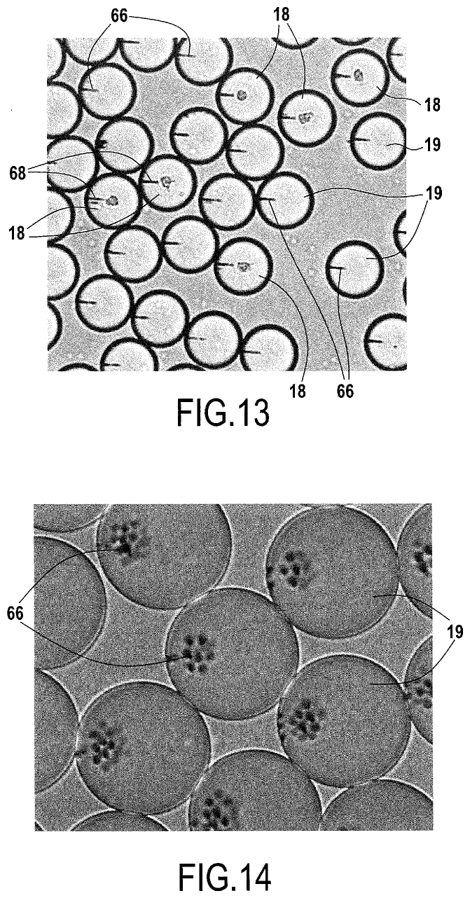

Overall, the process and apparatus according to the invention allow obtaining images such as shown in FIG. 13 of several droplets in a storage chamber. The image of FIG. 13 is a bright-field image showing droplets 19 without any cells, and assay droplets 18 containing cells. In all the droplets, an aggregate is clearly visible, perfectly in focus and located towards a side of the droplet. In assay droplets 18 containing cells, both the cell and the aggregate is clearly visible, perfectly in focus, and well separated one from the other. This ensures optimal and reliable detection of both the cell and the aggregate via known image analysis techniques.

It is to be noted that in the below described examples, the storage chamber 42 is part of the sample carrier device 44 and the magnetic field generator 70 is affixed to said sample carrier device. However, the magnet assembly does not need to be affixed to the sample carrier 44. The application of a magnetic field is especially important during the detection phase of the process, i.e. when the storage chamber is in front of the detector 60.

Therefore, the magnetic field generator may be independent of the sample carrier device 44. The magnetic field generator can be part of the detection station 62 of the apparatus. Indeed, as the storage chamber 42 is for example part of the sample carrier 44 device, the apparatus may be designed such that the sample carrier device 44 is movable relative to the detection station 62 comprising the detector 60. The sample carrier device 44 can thus be brought, manually or automatically, in coincidence with the detection station 62 for the performance of the detection phase. This coincidence corresponds to a detection position of the storage chamber at the detection station where the detection phase can be performed by the detector. In such a case, it can be provided that the magnetic field generator 70 can be fixed with respect to the detection station 62, especially fixed with respect to the detector 60. It is then preferable that, for the performance of the detection phase at the detection station, the storage chamber 42 is always brought into a predefined position with respect to station 62, especially with respect to the detector 60. In both cases, the apparatus is such that the magnetic field generator is configured to generate, at least when the storage chamber is at the detection station in the detection position where the detection phase can be performed by the detector, a magnetic field which is such that the paramagnetic particles are attracted horizontally towards at least one side of the storage chamber and vertically towards the bottom inner wall surface of the storage chamber, i.e. a magnetic field configured to attract the paramagnetic horizontally towards at least one side of the storage chamber and vertically towards the bottom inner wall surface of the storage chamber.

In the case shown in FIGS. 4, 5 and 6, the magnet assembly 70 comprises only one magnet 71, which is here a permanent magnet but which could also be an electromagnet. The magnet 71 exhibits a north pole N and a south pole S. The magnet is for example shaped as a parallelepiped. The magnet is for example arranged to extend along one side of the storage chamber 42.

In the example, the storage chamber 42 is elongated along a first direction of the horizontal plane. The magnet assembly, for example the single magnet 70, may thus be placed on a side of the storage chamber, transversally offset horizontally along a second direction, which is perpendicular to the first direction, compared to said side of the storage chamber.

In the shown example, the magnet assembly 70, for example the single magnet 71, may be entirely to the side of the storage chamber 42. In such a case, both of the poles of the magnet assembly are located horizontally to said side of the storage chamber. In such a case, the magnetic field is such that the paramagnetic particles are attracted, along the second direction, horizontally towards a side of the storage chamber.

It is to be noted that, in some embodiments, the assay droplets are preferably not able to move inside the storage chamber during the detection phase, i.e. when the magnetic field is applied. This may be achieved by substantially filing entirely the storage chamber with such assay droplets which are thus arranged contiguously over the whole extension of the storage chamber in the horizontal plane.

However, in other embodiments, the droplets may be able to move within the storage chamber. In this case, especially in the case where the magnetic field generator is not affixed to the storage chamber but pertains rather to the detection station, the application of the magnetic field may result in an initial movement of the magnetic field, due to the attraction of the paramagnetic particles contained in the droplets which may cause the movement of the droplets. However, even in that case, any movement of the droplets stops once the droplets have gathered on a corresponding side of the storage chamber.

To make sure that the paramagnetic particles are attracted towards an inferior portion of the stored assay droplets, the magnet assembly 70 is arranged below the level of the bottom inner wall surface 49 of the storage chamber 42. In the shown example, the bottom inner wall surface 49 of the storage chamber pertains to a bottom plate 48 of the storage chamber, for which it is a top surface. The bottom plate 48 exhibits a bottom external wall surface 51 opposite its inner wall surface 49. The magnet assembly is preferably arranged below the level of the bottom external wall surface 51.

In a non-depicted embodiment, it also could be provided that the paramagnetic particles are attracted towards a similarly disposed first lateral and inferior portion of a first group of the stored assay droplets, and towards a similarly disposed second lateral and inferior portion of a second group of the stored assay droplets, the first and second lateral and inferior portions being differently located in the respective assay droplets.

In the embodiment of FIGS. 4 to 6, the north pole N of the magnet assembly is on a top side of the magnet assembly 70, while the south pole S in on a bottom side of the magnet assembly, the magnet having thus a vertical magnetic moment. However, an inverted configuration is possible with the north pole N on top, as in the embodiment of FIGS. 11A and 11B, with still exhibits a vertical magnetic moment. In both cases, this would result in the aggregate being in the shape of rod-like elements in the horizontal as shown in FIGS. 7B, 7C, 8B, 8C, 8D and 13.

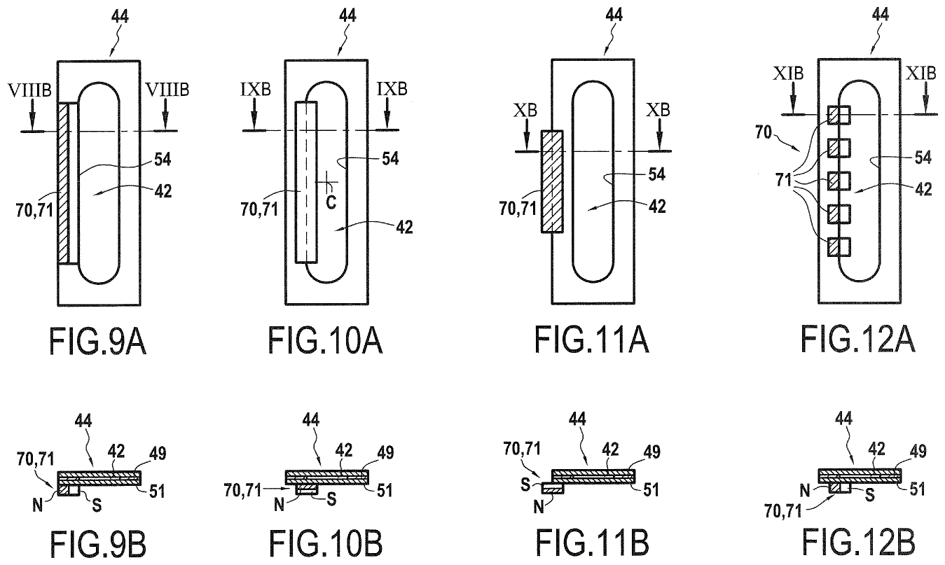

In the embodiment of FIGS. 9A and 9B, the north pole N of the magnet assembly is on one lateral side of the magnet assembly 70, while the south pole S in on the opposite lateral side of the magnet assembly according to the horizontal direction, thus having a horizontal magnetic moment. In such a case, this would result in the aggregate being as shown in FIG. 14 in a horizontal plane.

In some embodiments, such as those of FIG. 4 and of FIG. 4B, the side of the storage chamber 42 is defined as anything which is laterally offset with respect to a periphery of the storage chamber along the horizontal plane, for example the contour 54 of internal cut-out 52. In this case, the magnet assembly 70 does not overlap with the extension of the storage chamber 42.

In some embodiments, such as the embodiment of FIGS. 10A and 10B, the side of the storage chamber 42 is defined as anything which is laterally offset with respect to a centroid C of the extension of the storage chamber 42 along the horizontal plane. In this case, the magnet assembly 70 may overlap, but only partially, with the extension of the storage chamber 42.

In the previous examples, the magnet assembly 70 is continuous along one side of the storage chamber, over the entire extension of said side of the storage chamber. However, as in the example of FIGS. 11A and 11B, the magnet assembly may extend along only a part, less than the entirety, of the extension of the side of the magnet assembly. Moreover, the magnet assembly may be discontinuous, for example comprising several magnets which are spaced one from the other. FIGS. 12A and 12B show such arrangement, with several magnets spaced one from the other, in this case with magnets having a horizontal magnetic moment, and with magnets which partially overlap with the extension of the storage chamber. However, other configurations could have several spaced magnets with vertical magnetic moment, and/or several spaced magnets not overlapping with the extension of the storage chamber.

1. Microfluidic droplet-based assay process for analyzing biological cell activity by detection of at least one target analyte (16) contained in one or several droplets, wherein the process comprises the step of creating several assay droplets (18) of a liquid culture medium where an assay droplet contains:
paramagnetic particles (22, 22′);
detectable markers (24);
and, at least one biological cell (10) potentially capable of releasing at least one target analyte (16); wherein the paramagnetic particles (22, 22′) and the detectable markers (24) are able to bind with a target analyte (16) to form a marked and magnetized combination (25) with a target analyte (16),
wherein the process includes storing several assay droplets along a single layer along a horizontal storage plane,
wherein the process includes the generation of a magnetic field through the stored assay droplets for spatially arranging the paramagnetic particles inside the stored assay droplets,
and wherein the process includes detecting, within a given stored assay droplet, a physical characteristic of the detectable markers contained in the given assay droplet, by optical imaging of the detectable markers,
characterized in that the magnetic field induces the attraction of the paramagnetic particles towards a lateral and inferior portion of the given stored assay droplet in which they are contained.

2. Microfluidic droplet-based assay process according to claim 1, characterized in that the magnetic field is such that the paramagnetic particles (22, 22′) are attracted towards a similarly disposed lateral and inferior portion of all the stored assay droplets.

3. Microfluidic droplet-based assay process according to claim 1, characterized in that the magnetic field is asymmetric with respect to the stored assay droplets.

4. Microfluidic droplet-based assay process according to claim 1, characterized in that it comprises detecting fluorescence of fluorescent detectable markers and/or detecting radioisotope-labeled detectable markers.

5. Microfluidic droplet-based assay process according to claim 1, characterized in that it comprises providing, in the assay droplet, additional detectable markers able to bind with specific proteins expressed on the biological cell (10) surface to tag the biological cell (10).

6. Microfluidic droplet-based assay process according to claim 1, characterized in that the liquid culture medium comprises at least one vital dye and/or at least one non-vital dye.

7. Apparatus for a microfluidic droplet-based assay process for analyzing biological cell activity by detection of at least one target analyte (16) contained in one or several droplets, wherein the apparatus comprises a storage chamber (42) for storing several assay droplets (18) of a liquid culture medium containing:
paramagnetic particles (22, 22′);
detectable markers (24);
and, at least one biological cell (10) potentially capable of releasing at least one target analyte (16);
where the storage chamber is transparent in a detection process of the detectable markers and where the storage chamber is formed between two parallel top and bottom inner wall surfaces (47, 49) of the storage chamber, the inner wall surfaces extending parallel to a horizontal plane and being separated vertically by a height, such that the assay droplets are stored along a single layer;
wherein the apparatus includes a magnetic field generator (70, 71) for spatially arranging the paramagnetic particles inside the assay droplets stored in the storage chamber,
and wherein the apparatus includes a detector (60) of a physical characteristic of the detectable markers contained in
a given assay droplet stored in the storage chamber,
characterized in that the magnetic field generator generates a magnetic field such that the paramagnetic particles are attracted horizontally towards at least one side of the storage chamber and vertically towards the bottom inner wall surface of the storage chamber.

8. Apparatus according to claim 7, characterized in that the magnetic field generator comprises one or several magnets (71) forming a magnet assembly (70) which is arranged below the level of the bottom inner wall surface (49) of the storage chamber.

9. Apparatus according to claim 7, characterized in that the inner bottom wall surface (49) of the storage chamber pertains to a bottom wall (48) of the storage chamber which exhibits a bottom external wall surface (51) opposite its inner wall surface, and in that the magnet assembly is arranged below the level of the bottom external wall surface (51).

10. Apparatus according to claim 7, characterized in that the magnetic field generator (70, 71) generates a magnetic field such that the paramagnetic particles are attracted horizontally towards a same side of the storage chamber (42).

11. Apparatus according to claim 7, characterized in that the magnetic field generator comprises one or several magnets (71) forming a magnet assembly (70) and having each magnetic poles, wherein at least one of the poles of each of the magnets is located horizontally to a same side of the storage chamber (42).

12. Apparatus according to claim 7, characterized in that the magnetic field generator comprises one or several magnets (71) forming a magnet assembly (70) and having each magnetic poles, wherein both of the poles of the one or several magnets are located horizontally to a same side of the storage chamber.

13. Apparatus according to claim 7, characterized in that the storage chamber (42) is part of a sample carrier device (44) and in that the magnetic field generator (70, 71) is affixed to said sample carrier device.

14. Apparatus according to claim 7, characterized in that the magnetic field generator (70, 71) is part of a detection station (62) of the apparatus and in that the storage chamber (42) is part of a sample carrier device (44), the detection station and the sample carrier device being movable relative one to the other and being brought into coincidence for detection of at least one target analyte contained in one or several droplets contained in the storage chamber of the sample carrier device.

15. Apparatus according to claim 7, characterized in that the sample carrier device (44) comprises two plates (46, 48) forming the two walls of the storage chamber (42), and in that the two plates are affixed one to the other by an adhesive film (50) sandwiched between the two plates, wherein the thickness of the adhesive film determines vertically the height of the storage chamber.

16. Apparatus according to claim 15, characterized in that the adhesive film (50) comprises an internal cutout (52), the border (54) of which delimits the extension of the storage chamber (42) along the horizontal plane.
